(19) United States
(12) Patent Application Publication
(10) Pub. No.: US 2025/0082479 A1
(43) Pub. Date: Mar. 13, 2025

(54) ANTI-RETROPULSION LUMBAR INTERBODY DEVICE

(71) Applicant: Dereck PEERY, ARDMORE, OK (US)

(72) Inventor: Dereck PEERY, ARDMORE, OK (US)

(21) Appl. No.: 18/439,155

(22) Filed: Feb. 12, 2024

Related U.S. Application Data
(60) Provisional application No. 63/537,750, filed on Sep. 11, 2023.

(51) Int. Cl. A61F 2/44; A61F 2/30

(52) U.S. Cl. CPC A61F 2/4455; A61F 2002/30112; A61F 2002/30433; A61F 2002/30579; A61F 2002/30845

(57) ABSTRACT

An anti-retropulsion lumbar interbody device has a spacer body positionable, from a posterior direction, between a first vertebrae and a second vertebrae. At least one fixation member is connected to the spacer body. The at least one fixation member is movable between a retracted position and a deployed position, wherein in the deployed position, at least a portion of the at least one fixation member extends from the spacer body to a position configured to contact the first or second vertebrae when the spacer body is positioned between the first and second vertebrae. Wherein after the at least one fixation member is in the deployed position, and when the spacer body is moved in an anterior direction, the at least one fixation member self-retracts, thereby allowing the spacer body to be removed from the position between the first and second vertebrae.

### CROSS REFERENCE TO RELATED APPLICATION

This application claims benefit of U.S. Provisional Application Ser. No. 63/537,750 entitled, “Anti-Retropulsion Lumbar Interbody Device” filed Sep. 11, 2023, the entire disclosure of which is incorporated herein by reference.

### FIELD OF THE DISCLOSURE

The present disclosure is generally related to lumbar interbody devices and more particularly is related to anti-retropulsion lumbar interbody devices.

### BACKGROUND OF THE DISCLOSURE

Spinal fusion surgical procedures are routinely used to correct problems with the vertebrae. These procedures typically involve inserting an implant device between adjacent vertebrae such that bone growth between the neighboring vertebrae fuses the vertebrae together to form a single, solid bone.

One particular spinal fusion procedure uses an implant device called an interbody. In interbody fusion procedures, a surgeon gains access to the intervertebral disc between the vertebrae. All or part of the intervertebral disc is removed from the disc space between two vertebrae until there is adequate space for insertion of the interbody. A purpose of the interbody is to provide support to the anterior column of the spine, such that when the interbody is in place between the two vertebrae, it acts as a physical spacer between the two vertebrae, preventing collapse of the intervertebral disc space. Additionally, the interbody is intended as a permanent implant, and it serves as a carrier for graft material. It is commonly formed with cavities, holes, or textured surfaces which interface with graft material, thereby allowing for bone growth to envelope the interbody as the two vertebrae fuse.

There are various types of interbody fusion procedures. Posterior lumbar interbody fusions (PLIF) is a procedure where the surgeon gains access to the vertebrae from a posterior position, e.g., from the back of the patient. While posterior access provides direct access to the spine, a surgeon typically needs to remove portions of the neural arch or vertebral arch of the vertebrae to gain access to the nerve root and spinal cord. The surgeon then delicately retraces the nerve root and spinal cord laterally, such that access to the intervertebral disc is achieved. Transforaminal lumbar interbody fusions (TLIF) is a variation of PLIF, where access to the vertebrae is achieved more from the side. This approach requires less movement of the nerve root and spinal cord. Anterior lumbar interbody fusion (ALIF) is a procedure where a surgeon gains access to the vertebrae from the anterior position, e.g., from the front of the patient, commonly through an incision in the abdomen of the patient. ALIF permits access to the intervertebral disc without needing to move the nerve root and spinal cord positioned along the posterior of the spine.

FIG. 1 is an illustration of a vertebrae of the spine 10 along the lumbar, in accordance with the prior art, where each vertebrae 12 is positioned with an intervertebral disc 14 therebetween. The nerve root and spinal cord 16 is positioned along a posterior position 18 of the spine 10 while the anterior position 20 is on the opposing side of the spine 10. As can be seen in FIG. 1, one of the intervertebral discs 14 has a decreased height, such that treatment through PLIF, TLIF, or ALIF may be used to restore disc height.

A known and significant complication that occurs after these surgeries is a condition called retropulsion of the interbody. This is when the interbody backs out of the disc space posteriorly and into the nerve root causing severe radicular pain to the patient. The only current mechanism to prevent the retropulsion of the implant is by “press fitting” the implant. In this technique, the implant is equipped with small, backward facing grooves to try and diminish the chances of retropulsion of the interbody occurring. However, this technique still falls short of providing an effective and reliable method to prevent retropulsion of the interbody.

Thus, a heretofore unaddressed need exists in the industry to address the aforementioned deficiencies and inadequacies.

### SUMMARY OF THE DISCLOSURE

Embodiments of the present disclosure provide a system and method for an anti-retropulsion lumbar interbody device. Briefly described, in architecture, one embodiment of the system, among others, can be implemented as follows. An anti-retropulsion lumbar interbody device has a spacer body positionable, from a posterior direction, between a first vertebrae and a second vertebrae. At least one fixation member is connected to the spacer body. The at least one fixation member is movable between a retracted position and a deployed position, wherein in the deployed position, at least a portion of the at least one fixation member extends from the spacer body to a position configured to contact the first or second vertebrae when the spacer body is positioned between the first and second vertebrae. Wherein after the at least one fixation member is in the deployed position, and when the spacer body is moved in an anterior direction, the at least one fixation member self-retracts, thereby allowing the spacer body to be removed from the position between the first and second vertebrae.

The present disclosure can also be viewed as providing methods of using an anti-retropulsion lumbar interbody device. In this regard, one embodiment of such a method, among others, can be broadly summarized by the following steps: locating a spacer body between a first vertebrae and a second vertebrae; removing the spacer body from between the first vertebrae and the second vertebrae by moving the spacer body in an anterior direction from an anterior position; self-retracting at least one fixation member when the spacer body is moved in the anterior direction from the anterior position.

The present disclosure can also be viewed as providing a system for spacing vertebrae. Briefly described, in architecture, one embodiment of the system, among others, can be implemented as follows. A spacer body is located between a first vertebrae and a second vertebrae, wherein the spacer body is positioned surgically from a posterior of a patient. At least one fixation member is connected to the spacer body and positioned in a deployed position, wherein when in the deployed position, at least a portion of the at least one fixation member is extended from the spacer body and is in contact with the first vertebrae or second vertebrae. Movement of the spacer body in an anterior direction from the deployed position causes the at least one fixation member to self-retract, thereby allowing the spacer body to be removed from between the first vertebrae and second vertebrae.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is an illustration of a vertebrae of the spine along the lumbar, in accordance with the prior art.

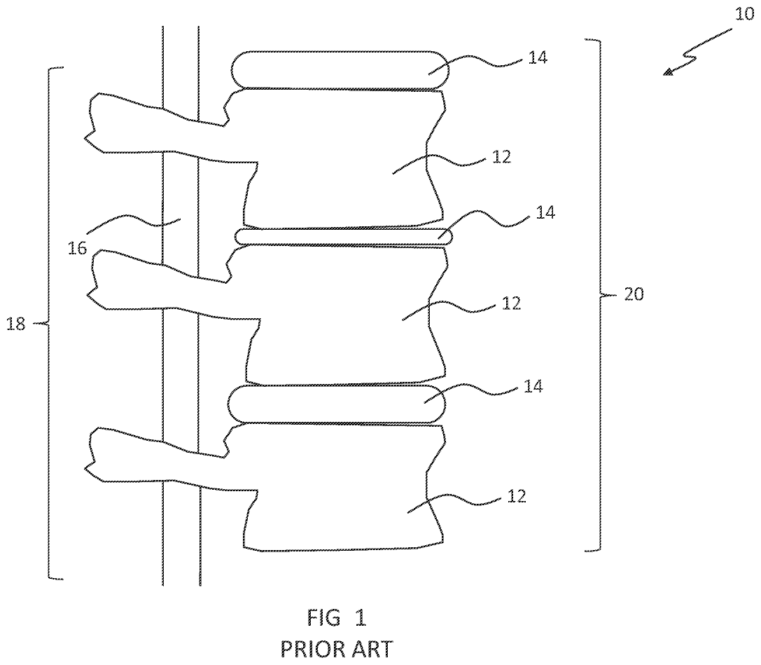

FIG. 2A is a top-view illustration of an anti-retropulsion lumbar interbody device in a retracted position, in accordance with exemplary embodiments of the present disclosure.

FIG. 2B is a side-view illustration of an anti-retropulsion lumbar interbody device in a retracted position, in accordance with exemplary embodiments of the present disclosure.

FIG. 2C is a side-view illustration of an anti-retropulsion lumbar interbody device in a deployed position, in accordance with exemplary embodiments of the present disclosure.

FIG. 3A is a top-view illustration of an anti-retropulsion lumbar interbody device in a retracted position, in accordance with exemplary embodiments of the present disclosure.

FIG. 3B is a side-view illustration of an anti-retropulsion lumbar interbody device in a retracted position, in accordance with exemplary embodiments of the present disclosure.

FIG. 3C is a side-view illustration of an anti-retropulsion lumbar interbody device in a deployed position, in accordance with exemplary embodiments of the present disclosure.

FIG. 4A-4B are perspective illustrations of an anti-retropulsion lumbar interbody device in a deployed position, in accordance with exemplary embodiments of the present disclosure.

FIG. 5 is a side-view illustration of an anti-retropulsion lumbar interbody device and a tool, in accordance with exemplary embodiments of the present disclosure.

FIG. 6 is a side-view illustration of the anti-retropulsion lumbar interbody device of FIGS. 2A-2C in a retracted position, in accordance with exemplary embodiments of the present disclosure.

FIG. 7 is a side-view illustration of the anti-retropulsion lumbar interbody device of FIG. 6 in a deployed position, in accordance with exemplary embodiments of the present disclosure.

FIG. 8A-8C are side-view illustrations of the anti-retropulsion lumbar interbody device of FIGS. 6-7 in a partially removed position between the vertebrae, in accordance with exemplary embodiments of the present disclosure.

FIG. 9A-9B are side-view illustrations of an anti-retropulsion lumbar interbody device, in accordance with exemplary embodiments of the present disclosure.

FIG. 10 is a flowchart illustrating a method of using an anti-retropulsion lumbar interbody device, in accordance with exemplary embodiments of the present disclosure.

### DETAILED DESCRIPTION

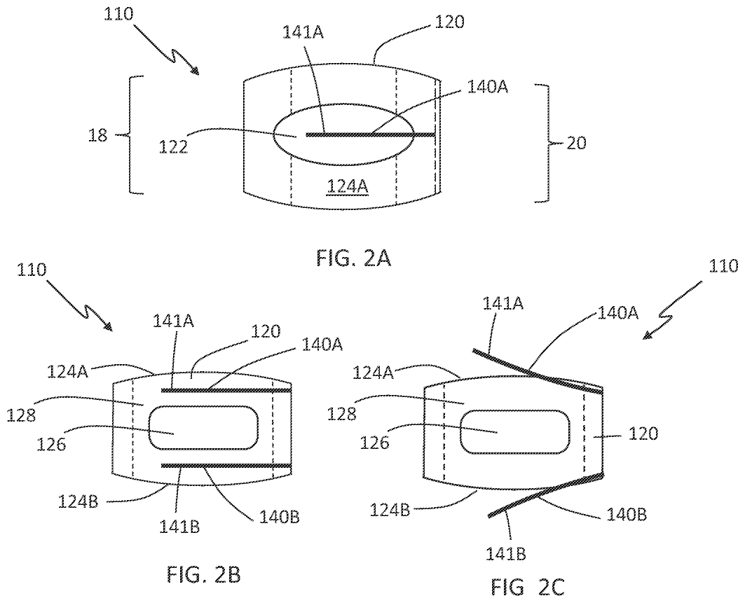

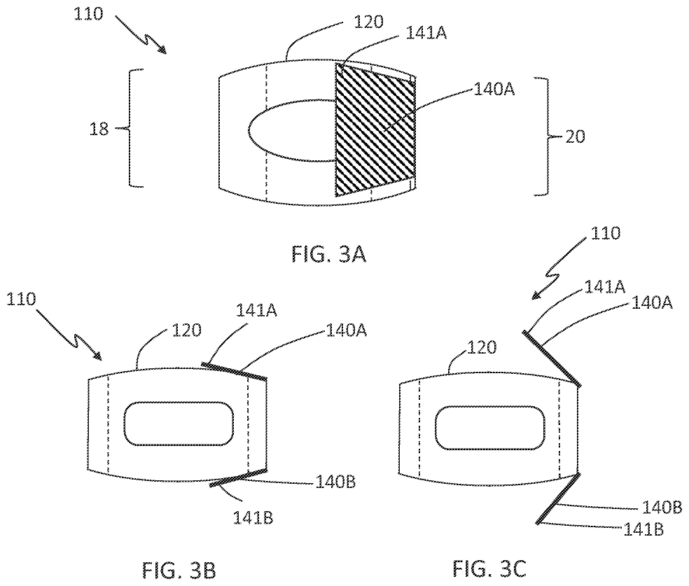

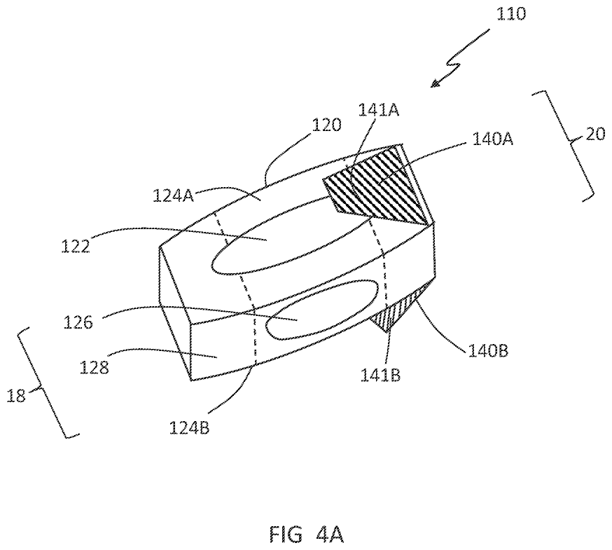

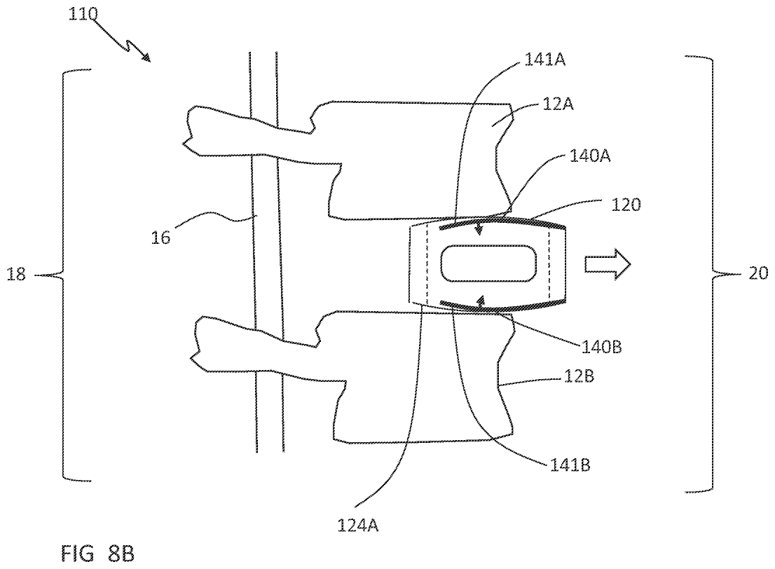

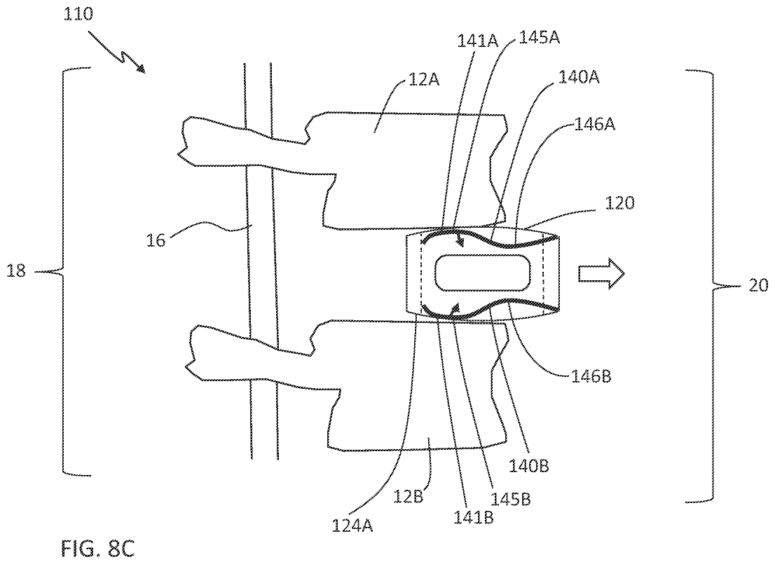

To improve over the shortcomings of conventional devices, an anti-retropulsion lumbar interbody device is provided. FIG. 2A is a top-view illustration of the anti-retropulsion lumbar interbody device in a retracted position, FIG. 2B is a side-view illustration of the anti-retropulsion lumbar interbody device in a retracted position, and FIG. 2C is a side-view illustration of the anti-retropulsion lumbar interbody device in a deployed position. FIGS. 6-7 are side-view illustrations of the anti-retropulsion lumbar interbody device of FIGS. 2A-2C in a retracted position (FIG. 6) and in deployed positions (FIG. 7).

As shown in FIGS. 2A-2C, the anti-retropulsion lumbar interbody device 110, which may be referred to as “interbody device 110” includes a spacer body 120 which generally forms the structure of the interbody device 110. The spacer body 120 may have a three-dimensional shape with at least a first aperture 122 positioned between a top surface 124A and a bottom surface 124B, and at least a second aperture 126 positioned between opposing sidewalls 128. The spacer body 120 may include various other features such as additional holes, cavities, or apertures, surface texturing or contours, textual markings, or other similar features.

At least one fixation member 140A is connected to the spacer body 120. As shown in FIGS. 2A-2C, the spacer body 120 has two fixation members 140A, 140B, where each of the fixation members 140A, 140B is connected to the spacer body 120 along a distal side thereof, and extends to a terminating portion of the fixation member 140A, 140B which is substantially positioned towards a midsection of the spacer body 120. It is noted that the at least one fixation member 140A may include any type of structure or combination of structures which can be used for fixing the spacer body 120 in a substantially stationary location in between two vertebrae during a spinal fusion procedure, or after completion of a spinal fusion procedure. For instance, the at least one fixation member 140A may include an elongated structure which is affixed at one end and free at another end, as is shown in FIGS. 2A-2C, which may be generally understood as a fin or set of fins which can extend from the spacer body 120. In other examples, the fixation member 140A may also include structures, such as hooks, protrusions, threaded features, grooved features, or other features which can perform the structural function of fixing the spacer body 120.

It is noted that the fixation members 140A, 140B may be oriented horizontally or vertically along the spacer body 120. The fixation members 140A, 140B can be located anywhere along the spacer body 120, including the anterior portion, the posterior portion, or a mid-portion. The fixation members 140A, 140B can be made of any metallic material, compound, or plastic. The size and length of the fixation members 140A, 140B may be dependent upon the size of the spacer body 120. The fixation members 140A, 140B may be equipped with serrations, grooves, or any other design to help with fixation.

The at least one fixation member 140A is movable between a retracted position and a deployed position. The retracted position may be characterized as a position where the fixation member 140A, or a plurality of fixation members 140A, 140B are generally positioned next to or proximate to the spacer body 120, such that the fixation members substantially conform to the footprint or shape of the spacer body 120 itself. In the retracted position, the spacer body 120 may be insertable between two vertebrae with ease, i.e., where the fixation members do not obstruct the insertion movement of the spacer body 120. The deployed position may be characterized as a position where at least one side, or one part of the fixation members 140A, 140B extend from the spacer body 120, such that the extending portion of the fixation members 140A, 140B are positioned removed from the spacer body 120. In one example, the deployed position may be where a substantial entirety of the fixation members 140A, 140B are positioned fully extended from the spacer body 120, such as above and below the top and surface planes of the spacer body 120. In this position, the free ends 141A, 141B of the fixation members 140A, 140B may be in a location where it will interface with the vertebrae adjacent to where the spacer body 120 has been positioned.

The deployed position may be characterized as a position where at least one side, or at least one part of the fixation members 140A, 140B extend from the spacer body 120 such that the extending portion of the fixation members 140A, 140B are positioned removed from the spacer body 120. In another example, the deployed position may be where a substantial entirety of the fixation members 140A, 140B are positioned fully extended from the spacer body 120, such as above and below the top and surface planes of the spacer body 120. In this position, the free ends 141A, 141B of the fixation members 140A, 140B may be in a location for interference and contact with the vertebrae adjacent to where the spacer body 120 has been positioned. Interfacing with the vertebrae may include any type of contact which allows the spacer body 120 to remain in a substantially fixed position between the two vertebrae.

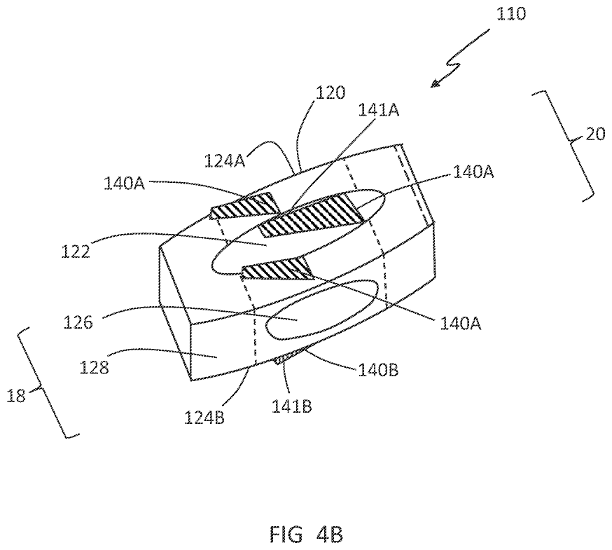

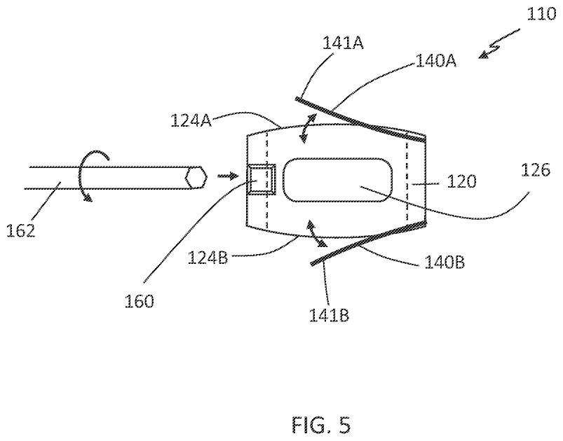

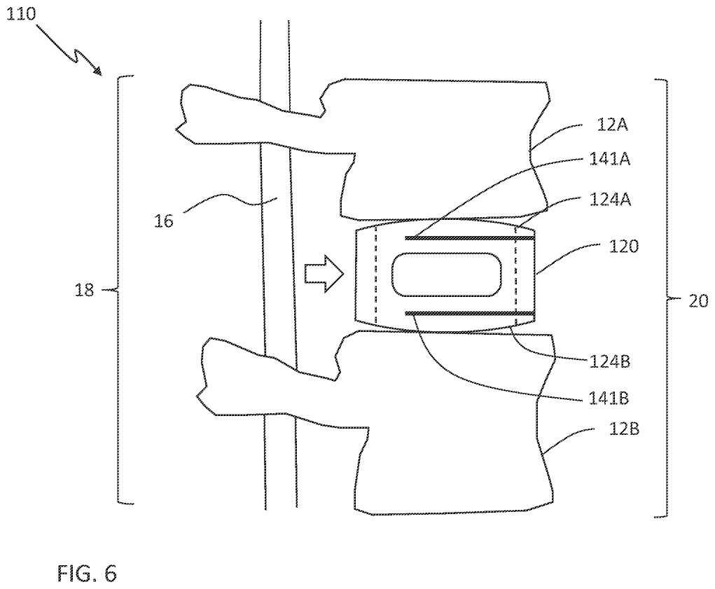

FIGS. 3A-3C provide another example of the interbody device 110 in accordance with embodiments of the present disclosure. In this example, the spacer body 120 is equipped with two fixation members 140A, 140B which are structured as fins having a planar surface. FIG. 3A illustrates a top-view of the interbody device 110 with the fin-like fixation member 140A, while FIGS. 3B-3C illustrate side views of the fin-like fixation members 140A, 140B. FIG. 3B illustrates the fin-like fixation members 140A, 140B in a retracted position where the fins largely conform to the shape of the spacer body 120, while FIG. 3C illustrates the fin-like fixation members 140A, 140B in a deployed position, extending above and below the spacer body 120.

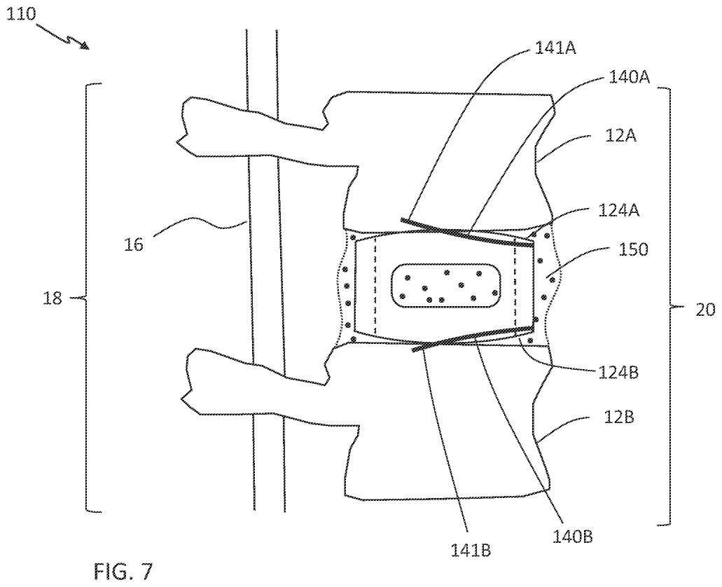

FIG. 4A-4B are perspective illustrations of the anti-retropulsion lumbar interbody device 110 in a deployed position, in accordance with exemplary embodiments of the present disclosure. FIG. 4A illustrates the interbody device 110 of FIG. 3C, with two fin-like fixation members 140A, 140B in a deployed position, extending above and below the spacer body 120. In some examples, the fin-like fixation members 140A, 140B may be bifurcated. In other examples, the fixation members 140A, 140B may be a structure with multiple extending prongs, where at least one of the prongs of the fixation members 140A, 140B contacts a vertebrae 12A, 12B (illustrated in FIG. 7). The two fin-like fixation members 140A, 140B may attach to a top surface 124A and a bottom surface 124B of the spacer body 120. The fixation members 140A, 140B may be positioned anywhere along the top surface 124A and the bottom surface 124B. In yet another example, the fixation members 140A, 140B are attached to the spacer body in a location at a corner of the top surface 124A and the bottom surface 124B. The fixation members 140A, 140B may connect at a corner, where the corner is defined by two substantially different planes of the spacer body 120 intersecting.

FIG. 4B illustrates another example of the interbody device 110 of FIG. 4A, having a plurality of fixation members 140A, 140B attached to the spacer body 120. In some examples, the fixation members 140A, 140B, may be attached at a midpoint of the top surface 124A and bottom surface 124B of the spacer body 120. In other examples, the plurality of fixation members 140A, 140B may be attached on each side of the top surface 124A and bottom surface 124B, where each side is a substantially flat plane between the first aperture 120 and an edge sidewall of each of the top surface 124A and bottom surface 124B.

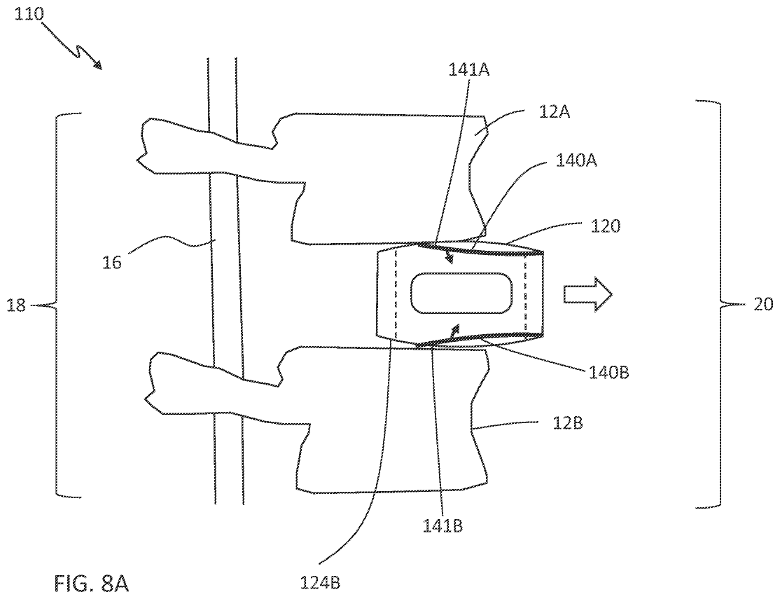

While the interbody device 110 has fixation members 140A, 140B which self-retract from movement of the spacer body 120 in the anterior direction, there are various techniques which may be used to allow the fixation members 140A, 140B to be deployed. Any type of mechanical deployment device, system, or structure may be used to actuate part of the interbody device 110 to deploy the fixation members 140A, 140B, as may be known in the art, whereby the fixation members 140A, 140B may be manually deployed, spring loaded, or even fixed. In one example, as shown in FIG. 5, which is a side-view illustration of an anti-retropulsion lumbar interbody device and a tool, in accordance with exemplary embodiments of the present disclosure, the interbody device 110 may include a deployment fixation member releasing mechanism 160 which is positioned on the spacer body 120, commonly on a posterior side thereof, where a tool 162 or implement can be used to activate the fixation member releasing mechanism 160 to cause the distal or free ends 141A, 141B of the fixation members 140A, 140B to be deployed. As such, when the spacer body 120 is in the desired position, the surgeon may use the tool 162 to contact the posterior portion of the spacer body 120 to activate the fixation member releasing mechanism 160, thereby allowing the fixation members 140A, 140B to deploy. Such mechanical structures of the fixation member releasing mechanism 160 may include the use of threaded elements, springs, friction-fit elements, or similar features. As the spacer body 120 is surgically inserted, the fixation member releasing mechanism 160 may be facing posteriorly, such that it is visible and accessible by the surgeon until the surgical site is closed. The fixation member releasing mechanism 160 may be found only on a posterior side, as the removal of the spacer body 120 from an anterior direction may not necessitate mechanical actuation of the fixation member releasing mechanism 160 by a tool 162, as the fixation members 140A, 140B self-retract when moved in an anterior direction.

In FIG. 6, the spacer body 120 is positioned in a location between the first and second vertebrae 12A, 12B, where the top and bottom surfaces 124A, 124B of the spacer body 120 are positioned contacting or close to the top and bottom surfaces of the first and second vertebrae 12A, 12B. The spacer body 120 achieves this position by being moved from a posterior direction into the spinal column. For example, the surgeon may access the patient's spinal column from the posterior 18, or rear side of the patient, through an incision in the patient's back. The surgeon may then remove portions of the neural arch of the vertebrae to gain access to the disc which is damaged. The surgeon then moves the nerve root 16 in the lateral direction to gain unimpeded access to the disc, such that the disk can be removed and the spacer body 120 can be inserted.

In the example shown in FIG. 6, the spacer body 120 is inserted in a direction with the affixed sides of the fixation members 140A, 140B in the forward part of the spacer body 120. When the spacer body 120 is fully in position between the vertebrae 12A, 12B, the fixation members 140A, 140B may be deployed, as shown in FIG. 7. Here, the surgeon may actuate or activate the interbody device 110 to cause the fixation members 140A, 140B, or part thereof, to expand outwards, generally past the top and bottom surfaces 124A, 124B of the spacer body 120. In this position, the exposed or extended portions of the fixation members 140A, 140B can contact the vertebrae 12A, 12B to fix the spacer body 120 in a substantially stationary position between the vertebrae 12A, 12B. In this position, bone growth 150 is permitted to be formed between the vertebrae 12A, 12B, where the spacer body 120 is substantially encapsulated within the bone growth 150. The final desired outcome is a fully fused first vertebrae 12A to the second vertebrae 12B with the spacer body 120 therein.

In many instances of spinal fusion surgery, placement of an interbody between vertebrae can be achieved successfully and can provide long term, positive results for the patient. However, in some situations, complications are experienced. For example, in posterior lumbar fusion procedures, one common complication is the interbody moving after it is positioned between the vertebrae, and in particular moving in a posterior direction towards the nerve root. Contact with the nerve root can cause severe radicular pain to the patient. The interbody device 110 is designed to prevent movement of the spacer body 120 after a surgical process is complete. Conventional interbody devices may use fixation members for permanent fixation, e.g., where bone growth is intended to encapsulate the fixation members such that the bones fuse to the fixation members. In contrast, the fixation members 140A, 140B of the subject disclosure are utilized and intended for stability purposes, to prevent movement of the spacer body 120 in a posterior direction, where it could contact the nerve root 16. As such, the fixation members 140A, 140B are oriented to prevent posterior movement of the spacer body 120, but they do not prevent other movement of the spacer body 120, nor are they intended to be used as permanent fixation members.

Another possible complication with spinal fusion procedures is when bone growth is not successful, and the bones of the patient do not grow through the holes and apertures within the interbody. This situation happens in approximately 8 to 10% of all PLIF procedures.

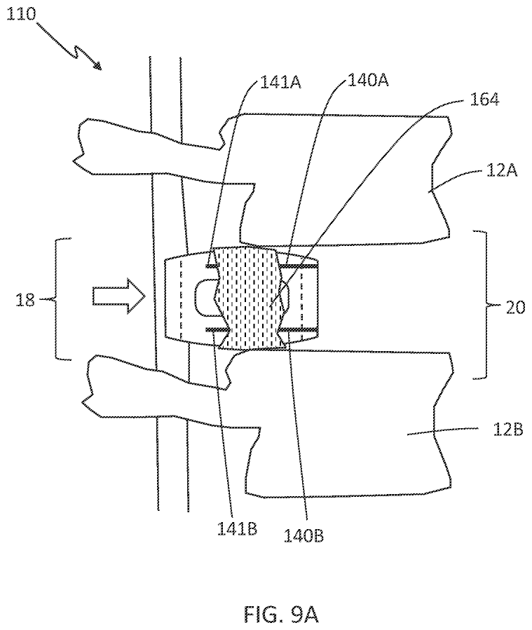

In these situations, revisions to the placement of the interbody are often needed. Even when an interbody is initially placed from a posterior direction, e.g., through the patient's lower back, a correction to the placement of the interbody is often done from an anterior direction from an incision on the patient's lower abdomen. The interbody device 110 permits convenient revisions to the positioning thereof, since it allows for a fixation member 140A, 140B which has been deployed, and therefore is in the deployed position, to be self-retracting when the spacer body 120 is moved in an anterior direction. Self-retracting of the fixation members 140A, 140B may be understood as automatic retraction of the fixation members 140A, 140B which is achieved through only anterior movement of the spacer body 120, without the need for mechanical actuation or manipulation of the interbody device 110, apart from the pulling force towards the anterior direction. This ability allows the spacer body to be removed from the position between the first and second vertebrae 12A, 12B without the need to mechanically retract the fixation members 140A, 140B. FIGS. 8A-8C depicts the movement of the spacer body 120 in the anterior direction, where the fixation members 140A, 140B self-retract to a position where they do not inhibit or substantially inhibit anterior movement of the spacer body 120.

FIGS. 8A-8C are side-view illustrations of the anti-retropulsion lumbar interbody device of FIGS. 6-7 in a partially removed position between the vertebrae, in accordance with exemplary embodiments of the present disclosure. In particular, FIGS. 8A-8C further depict possible additional shapes the fixation members 140A, 140B which may be used, as dependent on the design of the interbody device 110. As shown, FIG. 8A depicts the fixation members 140A, 140B self-retracting to a position where they do not inhibit or substantially inhibit anterior movement of the spacer body 120, where the fixation members 140A, 140B are exteriorly concave in shape. In this example, the use of the exteriorly concave shape for the fixation members 140A, 140B allows the distal or free ends 141A, 141B of the fixation members 140A, 140B to contact the vertebrae 12A, 12B. In the example of FIG. 8A, the exteriorly concave shape of the fixation members 140A, 140B may cause more pressure to be exerted on the vertebrae 12A, 12B by the distal or free ends 141A, 141B.

FIG. 8B depicts the fixation members 140A, 140B self-retracting to a position where they do not inhibit or substantially inhibit anterior movement of the spacer body 120, where the fixation members 140A, 140B are exteriorly convex in shape. In this example, the use of the exteriorly convex shape for the fixation members 140A, 140B allows a middle section of the fixation members 140A, 140B to contact the vertebrae. The middle section of the fixation members 140A, 140B may provide a greater surface area to contact with the vertebrae 12A, 12B, thus potentially increasing the amount of friction between the fixation members 140A, 140B and the vertebrae 12A, 12B, which may further secure the spacer body 120 in the desired position.

FIG. 8C depicts the fixation members 140A, 140B self-retracting to a position where they do not inhibit or substantially inhibit anterior movement of the spacer body 120, where the fixation members 140A, 140B contain an exteriorly convex segment 145A, 145B and/or an exteriorly concave segment 146A, 146B. In this example, the use of the exteriorly convex segment 145A, 145B coupled with an exteriorly concave segment 146A, 146B may create greater surface area at an inflection point of the exteriorly convex segment 145A, 145B while the exteriorly concave segment 146A, 146B may create a greater force to press the inflection point of the exteriorly convex segment 145A, 145B onto the vertebrae 12A, 12B. Furthermore, in this example, the inflection point of the exteriorly convex segment 145A, 145B may provide a greater surface area for the distal or free end 141A, 141B of the fixation members 140A, 140B to contact the vertebrae 12A, 12B.

In another example, all or part of the fixation members 140A, 140B, once deployed, could be permanently deployed, self-retracting, or mechanically retracted. For instance, a portion of the fixation elements may be permanently deployed while others retract.

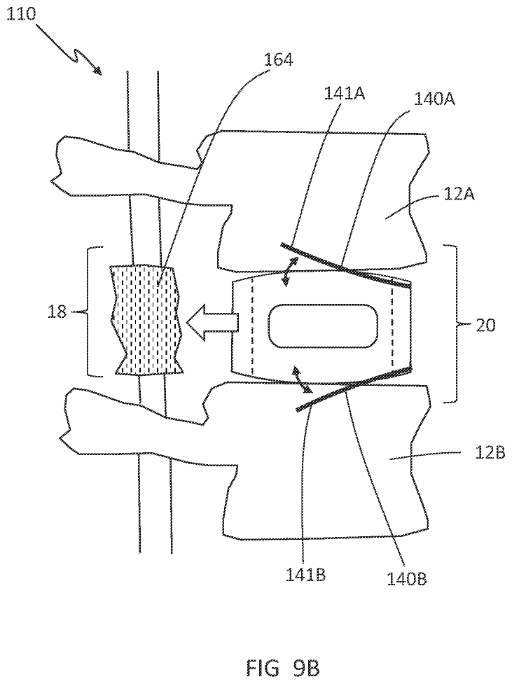

In another example, as shown in FIGS. 9A-9B, sleeve 164 or cap may be positioned over the spacer body 120 and hold the fixation members 140A, 140B in the retracted position while the interbody device 110 is being positioned between the vertebrae 12A, 12B, as shown in FIG. 9A. When the interbody device 110 achieves the desired position between the vertebrae, the sleeve 164 may be removed, thereby allowing the fixation members 140A, 140B to move to the deployed position, as shown in FIG. 9B. The use of a sleeve 164 as a fixation member releasing mechanism 160 further reduces manufacturing and implantation costs, as in this example the fixation members 140A, 140B are held in the retracted position by the sleeve 164, and the fixation members 140A, 140B are deployed once the surgeon removes the sleeve 164 from the spacer body 120. The surgeon may use a tool 162 to assist in the removal of the sleeve 164 from the spacer body 120. The tool 162 may be any surgical tool 162 which can be inserted into an intervertebral space to remove the sleeve 164 from the spacer body 120 such that the fixation members 140A, 140B deploy upon at least partial removal of the sleeve 164.

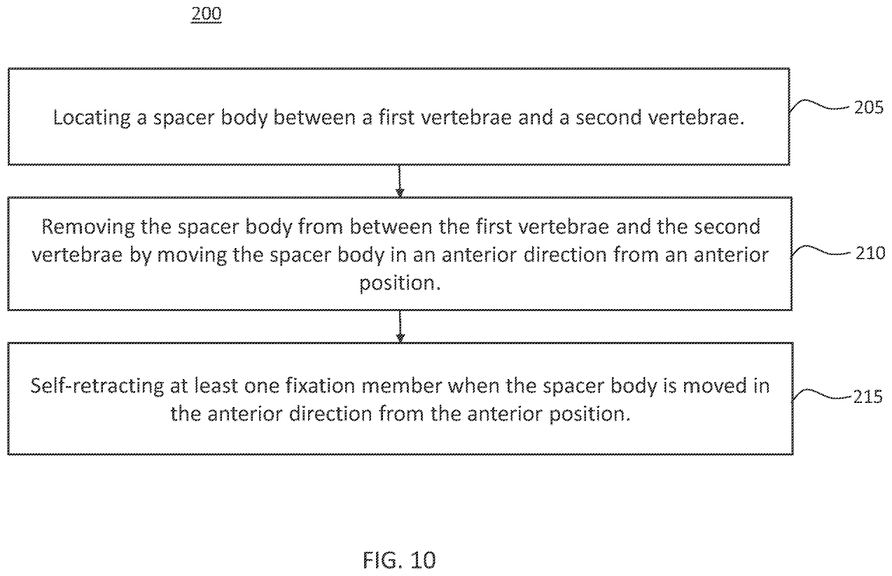

FIG. 10 is a flowchart 200 illustrating a method for using the interbody device 110 in accordance with the exemplary embodiments of the disclosure. It should be noted that any process descriptions or blocks in flow charts should be understood as representing modules, segments, portions of code or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

Method step 205 has the steps of locating a spacer body between a first vertebrae and a second vertebrae. At step 210 the spacer body is removed from between the first vertebrae and the second vertebrae by moving the spacer body in an anterior direction from an anterior position. At step 215, at least one fixation member self-retracts when the spacer body is moved in the anterior direction from the anterior position.

In furtherance of the described method, and with references to FIGS. 2A-10, in order to remove the spacer body 120 after locating the spacer body 120, a surgeon may cut into the patient anteriorly 18. Once the surgeon has cut anteriorly 18 and has surgically retracted portions of anatomy required to access the spine, the spacer body 120 located between the first vertebrae 12A and the second vertebrae 12B should be accessible. The surgeon may apply a force in the anterior direction 18 to the spacer body 120, whereby applying the force causes the at least one fixation member 140A, 140B to self-retract by moving in a direction towards the spacer body 120. The force applied may be a pulling force, where the surgeon pulls the spacer body 120 anteriorly into and out of the patient's abdominal cavity. The self-retracting property of the at least one fixation member 140A, 140B is a result of the at least one fixation member 140A, 140B being in between, and thus in contact with at least one of the first vertebrae 12A or second vertebrae 12B. Contact is between at least one of the first vertebrae 12A or second vertebrae 12B and an outside surface of the at least one fixation member 140A, 140B.

Additionally, a method to use the interbody device 110 may include the process of inserting the spacer body 120 between the first vertebrae 12A and second vertebrae 12B. The spacer body 120 may be inserted from a posterior 18 position. Once the surgeon has cut the patient from a posterior 18 position and has surgically retracted portions of anatomy to access at least one of the first vertebrae 12A or second vertebrae 12B, the spacer body 120 may be inserted from the posterior 18 position. During insertion from the posterior 18 position, the at least one fixation member 140A, 140B may be in a retracted position. The fixation members 140A, 140B may be deployed or actuated by a surgeon once the spacer body 120 is positioned in the desired location between the first vertebrae 12A and second vertebrae 12B. Deploying the fixation members may be done by the surgeon actuating the fixation members 140A, 140B by inserting a tool 162 into the fixation member releasing mechanism 160. Fully deploying the fixation members 140A, 140B may cause the fixation members 140A, 140B, or part thereof, to expand outwards, generally past the top and bottom surfaces 124A, 124B of the spacer body 120. In this position, the outer surfaces of the extended portions of the fixation members 140A, 140B can be contacting the vertebrae 12A, 12B, fixing the spacer body 120 in a substantially stationary position between the vertebrae 12A, 12B.

In some examples, the spacer body 120 may have a sleeve 164 or cap as the fixation member releasing mechanism 160 as depicted in FIGS. 9A-9B. When a sleeve 164 is used, it may be possible to hold the fixation members 140A, 140B in the retracted position by the sleeve 164 or cap while positioning the interbody device 110 between the vertebrae 12A, 12B. Removing the sleeve 164 or cap after the interbody device 110 is positioned between the vertebrae 12A, 12B in the desired position allows for the deploying of the fixation members 140A, 140B.

Forming bone growth 150 between the vertebrae 12A, 12B occurs once the outer surfaces of the extended portions of the fixation members 140A, 140B are contacting the vertebrae 12A, 12B, and the spacer body 120 is in a substantially stationary position between the vertebrae 12A, 12B. Substantially encapsulating the spacer body 120 within the bone growth 150 may occur, leading to the final desired outcome of fully fusing the first vertebrae 12A to the second vertebrae 12B with the spacer body 120 therein.

In addition to the interbody device 110 as described herein, it should be understood that the functionality described encompasses methods of using the interbody device 110, all of which are considered within the scope of the present disclosure.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

What is claimed is:

1. An anti-retropulsion lumbar interbody device comprising:
a spacer body positionable between a first vertebrae and a second vertebrae;
at least one fixation member connected to the spacer body, the at least one fixation member movable between a retracted position and a deployed position, wherein in the deployed position at least a portion of the at least one fixation member is extendable from the spacer body to contact the first or second vertebrae when the spacer body is positioned between the first vertebrae and second vertebrae; and
wherein when the spacer body is moved in an anterior direction from the deployed position, the at least one fixation member is self-retractable, thereby allowing the spacer body to be removed from between the first vertebrae and second vertebrae.

2. The anti-retropulsion lumbar interbody device of claim 1, further comprising:
a fixation member releasing mechanism located on a posterior side of the spacer body, wherein activation of the fixation member releasing mechanism deploys the at least one fixation member to the deployed position.

3. The anti-retropulsion lumbar interbody device of claim 2, wherein the fixation member releasing mechanism is a threaded fastener.

4. The anti-retropulsion lumbar interbody device of claim 2, wherein the fixation member releasing mechanism is a bias fastener.

5. The anti-retropulsion lumbar interbody device of claim 1, wherein at least an outer surface of the at least one fixation member is in contact with at least one of the first or second vertebrae.

6. The anti-retropulsion lumbar interbody device of claim 1, wherein the at least one fixation member has at least one of: an exteriorly convex shape, an exteriorly concave shape, an exteriorly convex segment, or an exteriorly concave segment.

7. The anti-retropulsion lumbar interbody device of claim 1, wherein the at least one fixation member self-retracts when the spacer body is moved in the anterior direction by contact between the at least one fixation member and the first vertebrae or the second vertebrae, thereby allowing the spacer body to be removed from the position between the first vertebrae and second vertebrae.

8. The anti-retropulsion lumbar interbody device of claim 1, wherein the at least one fixation member further comprises:
a first fixation member extendable outwards from a top surface of the spacer body; and
a second fixation member extendable outwards from a bottom surface of the spacer body, wherein the first and second fixation members self-retract when the spacer body is moved in the anterior direction.

9. A method for using an anti-retropulsion lumbar interbody device, the method comprising:
locating a spacer body between a first vertebrae and a second vertebrae;
removing the spacer body from between the first vertebrae and the second vertebrae by moving the spacer body in an anterior direction from an anterior position; and
self-retracting at least one fixation member when the spacer body is moved in the anterior direction from the anterior position.

10. The method for using an anti-retropulsion lumbar interbody device of claim 9, the method further comprising:
inserting a spacer body between the first vertebrae and second vertebrae from a posterior direction; and
deploying the at least one fixation member of the spacer body by moving at least a portion of the at least one fixation member outwards to contact the first vertebrae or the second vertebrae.

11. The method for using an anti-retropulsion lumbar interbody device of claim 9, wherein removing the spacer body further comprises:
cutting anteriorly into a patient;
applying a force in the anterior direction to the spacer body, whereby applying the force causes the at least one fixation member to self-retract by moving in a direction towards the spacer body.

12. The method for using an anti-retropulsion lumbar interbody device of claim 9, wherein self-retracting of the at least one fixation member further comprises contact between at least one of the first or second vertebrae and an outside surface of the fixation members.

13. The method for using an anti-retropulsion lumbar interbody device of claim 9, wherein removing the spacer body further comprises applying a force to the spacer body in the anterior direction.

14. A system for spacing vertebrae comprising:
a spacer body located between a first vertebrae and a second vertebrae, wherein the spacer body is positioned surgically from a posterior of a patient; and
at least one fixation member connected to the spacer body and positioned in a deployed position, wherein when in the deployed position, at least a portion of the at least one fixation member is extended from the spacer body and is in contact with the first vertebrae or second vertebrae, and wherein movement of the spacer body in an anterior direction from the deployed position causes the at least one fixation member to self-retract, thereby allowing the spacer body to be removed from between the first vertebrae and second vertebrae.

15. The system for spacing vertebrae of claim 14, wherein contact between the at least one fixation member and the first vertebrae or second vertebrae prevents movement of the spacer body in the posterior direction.

16. The system for spacing vertebrae of claim 14, further comprising:
a fixation member releasing mechanism located on a posterior side of the spacer body, wherein activation of the fixation member releasing mechanism deploys the at least one fixation member to the deployed position.

17. The system for spacing vertebrae of claim 16, wherein the fixation member releasing mechanism is a threaded fastener.

18. The system for spacing vertebrae as in claim 16, wherein the fixation member releasing mechanism is a bias fastener.

19. The system for spacing vertebrae as in claim 14, wherein the at least one fixation member has at least one of: an exteriorly convex shape, an exteriorly concave shape, an exteriorly convex segment, or an exteriorly concave segment.

20. The system for spacing vertebrae of claim 14, wherein the at least one fixation member self-retracts when the spacer body is moved in the anterior direction by contact between the at least one fixation member and the first vertebrae or the second vertebrae, thereby allowing the spacer body to be removed from the position between the first vertebrae and second vertebrae.
